(19) United States
(12) Patent Application Publication
(10) Pub. No.: US 2018/0177829 A1
(43) Pub. Date: Jun. 28, 2018

(54) METHOD FOR TREATING SPINAL CORD INJURY USING PSA-NCAM POSITIVE NEURAL CELLS

(71) Applicant: S-BIOMEDICS, Seoul (KR)

(72) Inventors: Dong Wook KIM, Seoul (KR); Han Soo KIM, Goyang-si (KR)

(21) Appl. No.: 15/390,878

(22) Filed: Dec. 27, 2016

(51) Int. Cl. A61K 35/30

(52) U.S. Cl. CPC A61K 35/30

(57) ABSTRACT

The present invention relates to a composition for preventing or treating spinal cord injury, the composition containing poly-sialylated neural cell adhesion molecule (PSA-NCAM) positive neural cells as an active ingredient. The PSA-NCAM positive neural cells (e.g., PSA-NCAM positive neural precursor cells) of the present invention, when transplanted into spinal cord injury animal models, are differentiated into astrocytes, oligodendrocytes, or neurons (NeuN positive cells), which are three types of important cells constituting neural tissues, and exhibiting a behavior improvement effect due to the recovery of neural functions and the restoration of the injured spinal cord tissues. Thus, the cells can be used for a composition for preventing, alleviating, or treating spinal cord injury.

### TECHNICAL FIELD

The present invention relates to a method for treating spinal cord injury using PSA-NCAM positive neural cells.

### BACKGROUND ART

Stem cells are regarded as a promising therapeutic candidate material for various diseases due to the multipotency thereof. For example, mesenchymal stem cells (MSCs) can be easily obtained and isolated from tissues and are known to release many bioactive factors that promote angiogenesis and inhibit inflammation (Caplan, A. I., & Dennis, J. E. (2006). Journal of Cellular Biochemistry, 98, 1076-1084). These characteristics of MSCs have been considered in studies for the application to the treatment of a number of human diseases. Recent studies have found that MSCs contribute to the tissue repair in a large number of animal models and human clinical settings (Chen, J., Li, Y., Katakowski, M., et al. (2003). Journal of Neuroscience Research, 73, 778-786; Kopen, G. C., Prockop, D. J., & Phinney, D. G. (1999). Proceedings of the National Academy of Sciences, 96, 10711-10716). Several reports proposed the in vitro differentiation ability of MSC into the neural lineage (Bae, K. S., Park, J. B., Kim, H. S., Kim, D. S., Park, D. J., & Kang, S. J. (2011). Yonsei Medical Journal, 52, 401-412) and astrocytes (Kopen, G. C., Prockop, D. J., & Phinney, D. G. (1999). Proceedings of the National Academy of Sciences, 96, 10711-10716), but there is no definite evidence as to what functions the differentiated cells perform in vivo. It seems that the favorable effect of MSCs is induced by paracrine mechanisms rather than cellular replacement, and therefore, the transplantation of MSCs would have temporary and limited effects but not the alleviation maintained for a long term (Cho, S. R., Kim, Y. R., Kang, H. S., et al. (2009). Cell Transplantion, 18, 1359-1368).

In contrast, embryonic stem cells (ESCs) may differentiate into all cell types derived from three embryonic germ layers, and have a strong self-renewal ability. Noticeably, neural precursor cells (NPCs) derived from ESCs, mainly differentiate into a neural lineage cell type including neural cells, astrocytes, and oligodendrocyte, and thus are considered to be a cell source for repair of brain tissues.

These cells secrete several factors for promoting the survival and proliferation of endogenous neural precursor cells (Capone, C., Frigerio, S., Fumagalli, S., et al. (2007) PLoSOne, 7, e373). However, it has not yet been known how NPCs differentiated from ESCs contribute to the improvement of functions after transplantation in diseases models. The present inventors have discovered that polysialic acid-neural cell adhesion molecule (PSA-NCAM) positive NPCs (NPC^{PSA-NCAM+}) are primitive NPCs isolated from neural rosettes of human ESCs and iPSCs [9]. These cells strongly expressed neural markers without contamination of non-neural cell populations (80-90% of total cells), and were propagated for multiple passages while retaining their characteristics as primitive cells in our culture condition. Therefore, the present inventors have tried to investigate the therapeutic potential of NPC^{PSA-NCAM+} derived from human ESCs using rat spinal cord injury models. Meanwhile, MSCs have been reported to have a neuroprotective action through anti-inflammatory, angiogenesis promoting and an endogenous neural cell recruiting activities in ischemic brain tissues of stroke animal models [10].

Reprogrammed stem cells refer to cells with pluripotency derived from somatic cells with no pluripotency by an artificial reprogramming procedure through various means, such as gene transduction/transcriptional factor introduction, chemical treatment, and growth factor treatment, and these reprogrammed stem cells are commonly called induced pluripotent stem cells (iPSCs) [11]. When compared with embryonic stem cells, these cells show very high similarity in terms of cell morphology, culture requirement, proliferation rate, gene expression patterns, chromosome variation patterns, pluripotency, and teratoma forming ability in immunodeficient mice. The differentiation ability of the induced pluripotent stem cells is comparable to that of embryonic stem cells, but NPCs differentiated therefrom or the contribution of NPCs to the improvement of functions after transplantation in spinal cord injury models has not been known.

The present inventors have tried to discover that human ESC-derived NPC^{PSA-NCAM+} can be used to treat spinal cord injury or various neuroinflammatory diseases related to the spinal cord injury as a promising therapeutic resource exhibiting a behavior recovery effect in spinal cord injury animal models.

Throughout the entire specification, many papers and patent documents are referenced and their citations are represented. The disclosure of the cited papers and patent documents are entirely incorporated by reference into the present specification, and the level of the technical field within which the present invention falls and the details of the present invention are explained more clearly.

### DETAILED DESCRIPTION OF THE INVENTION

### Technical Problem

The present inventors researched and endeavored to develop a radical treatment method for spinal cord injury, and as a result, verified that the injection of neural precursor cells expressing PSA-NCAM, which is the neural adhesion molecule, into a site of lesion leads to an efficient treatment of spinal cord neural tissue injury.

Accordingly, an aspect of the present invention is to provide a method for treating spinal cord injury.

Other purposes and advantages of the present invention will become more obvious with the following detailed description of the invention, claims, and drawings.

### Technical Solution

In accordance with an aspect of the present invention, there is provided a method for treating spinal cord injury, comprising administering a composition, which comprises poly-sialylated neural cell adhesion molecule (PSA-NCAM)-positive neural cells as an active ingredient, to a subject in need thereof.

The present inventors researched and endeavored to develop a radical treatment method for spinal cord injury, and as a result, verified that the injection of neural precursor cells expressing PSA-NCAM, which is the neural adhesion molecule, into a site of lesion leads to an efficient treatment of spinal cord neural tissue injury.

As used herein, the term “treatment” refers to: (a) suppressing the development of disease, disorder, or symptom; (b) reducing disease, disorder, or symptom; or (c) curing disease, disorder, or symptom. The composition of the present invention either suppresses the development of, inhibits, or reduces the symptom of spinal cord injury upon transplanting the neural precursor cells to a subject with spinal cord injury. Therefore, the composition of the present invention per se may be a composition for treating spinal cord injury, or the composition of the present invention may be applied as a treatment adjuvant by the administration thereof together with another composition for treating spinal cord injury. Therefore, as used herein, the term “treatment” or “treatment agent” includes a meaning of “treatment aid” or “treatment adjuvant”.

As used herein, the term “administration” or “administer” refers to the direct application of a therapeutically effective amount of the composition of the present invention to a subject, to thereby form the same amount thereof in the body of the subject. Therefore, the term “administer” includes the injection of an active ingredient (neural cells) around a site of lesion, and thus the term is used in the same meaning as the term “inject”.

As used herein, the term “therapeutically effective amount” refers to the content of cells, which is sufficient to provide a therapeutic or prophylactic effect to a subject to which the composition is to be administered, and thus the term has a meaning including “prophylactically effective amount”. As used herein, the term “subject” includes, but is not limited to, human, mouse, rat, guinea pig, dog, cat, horse, cow, pig, monkey, chimpanzee, beaver, or rhesus monkey. Specifically, the subject of the present invention is human.

According to the present invention, the poly-sialylated neural cell adhesion molecule (PSA-NCAM) positive neural cells are differentiated from pluripotent stem cells.

As used herein, the term “stem cells” is a generic term for undifferentiated cells before differentiation into respective cells constituting tissues, and the stem cells have an ability to be differentiated into particular cells by particular differentiation stimuli (environment). Unlike cell division-ceased terminally differentiated cells, the stem cells are capable of producing the identical cells through cell division (self-renewal), and have plasticity in differentiation, in which the stem cells are differentiated into particular cells by differentiation inducing stimuli and may be differentiated into other cell types under different environments or by different stimuli.

The stem cells used in the present invention are pluripotent stem cells that proliferate indefinitely in vitro and can be differentiated into various cells derived from all embryonic layers (ectoderm, mesoderm, and endoderm). More specifically, the pluripotent stem cells are embryonic stem cells, induced pluripotent stem cells (iPSCs), embryonic germ cells, or embryonic carcinoma cells.

The embryonic stem cells are derived from the inner cell mass (ICM) of the blastocyst, and the embryonic germ cells are derived from primordial germ cells present in 5-10 week-old gonadal ridges.

Induced pluripotent stem cells (iPSCs) are one type of pluripotent stem cells artificially derived from non-pluripotent cells (e.g., somatic cells) by inserting a particular gene imparting pluripotency therein. Induced pluripotent stem cells are considered to be the same as pluripotent stem cells (e.g., embryonic stem cells) since the induced pluripotent stem cells have highly similar stem cell gene and protein expression patterns, chromosomal methylation pattern, doubling time, embryoid body formation capacity, teratoma formation capacity, viable chimera formation capacity, hybridizability, and differentiability ability as embryonic stem cells.

Herein, the poly-sialylated neural cell adhesion molecule (PSA-NCAM) positive neural cells are construed as a meaning including all of PSA-NCAM positive neural precursor cells, a PSA-NCAM positive neuronal precursor, and differentiated PSA-NCAM positive neural cells.

According to an embodiment, the neural cells are PSA-NCAM positive neural precursor cells.

According to an embodiment of the present invention, the neural precursor cells (NPCs) are neural precursor cells before or after the stage of neural rosettes, which are formed by inducing the differentiation of pluripotent stem cells (e.g., embryonic stem cells or induced pluripotent stem cells) into neural lineage cells.

Herein, the poly-sialylated neural cell adhesion molecule (PSA-NCAM) positive neural cells may be separated from neural rosettes, which are differentiated from pluripotent stem cells through neural differentiation stimulation, by using an anti-PSA-NCAM-antibody. The term “neural rosettes” refers to neural stem cells at the initial stage of neural differentiation of human embryonic stem cells, and the neural rosettes have a cylindrical radial form. The neural rosettes are composed of cells expressing early neuroectodermal markers, such as Pax6 and Sox1, and may be differentiated into various neural cells and neuroglial cells.

The stimulation of neural differentiation may be differentiated by a method that is ordinarily conducted in the art, for example, serum-free media (Tropepe V et al., Neuron. 30:6578(2001)), fibroblast growth factors (FGFs), and treatment with morphogens, such as Wnt and retinoic acid (RA) (Ying Q L et al. Nat Biotechnol. 21:183186(2003)), but is not limited thereto.

Polyclonal antibodies or monoclonal antibodies may be used as the antibody. The antibodies against PSA-NCAM may be produced by the methods that are conventionally conducted in the art, for example, a fusion method (Kohler and Milstein, European Journal of Immunology, 6:511-519 (1976)), a recombinant DNA method (U.S. Pat. No. 4,816,56), or a phage antibody library method (Clackson et al, Nature, 352:624-628 (1991) and Marks et al, J. Mol. Biol., 222:58, 1-597 (1991)). A general procedure for antibody production is described in detail in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, N Y, 1991, the disclosure of which are incorporated herein by reference. For example, hybridoma cells producing monoclonal antibodies may be obtained by fusing immortal cell lines to antibody-producing lymphocytes, the technology for which has been well known to those skilled in the art, and can be easily conducted. The polyclonal antibodies may be obtained by injecting PSA-NCAM antigens into an appropriate animal, collecting antisera from the animal, and then isolating antibodies from the antisera using the known affinity technique.

As used herein to recite the PSA-NCAM, the term “antibody” refers to a an antibody specific to PSA-NCAM, and the antibody specifically binds to the PSA-NCAM protein, and includes a complete form of an antibody and an antigen binding fragment of the antibody molecule. The complete antibody has a structure having two full-length light chains and two full-length heavy chains, and the light chains are linked to the heavy chains via a disulfide linkage, respectively. The antigen-binding fragment of the antibody molecule is a fragment having an antigen binding function, and includes Fab, F(ab′), F(ab′)2, and Fv.

For the separation of PSA-NCAM-positive neural precursor cells using an antibody, fluorescence-activating cell sorters (FACS), magnetic activated cell sorter (MACS), antibody-coated plastic adherence, and complement-mediated lysis may be used.

As used herein, the term “spinal cord injury” includes both traumatic spinal cord injury and non-traumatic spinal cord injury. The traumatic spinal cord injury includes a flexion injury, vertical compression injury, hyperextension injury, or flexion rotation injury, but is not limited thereto. The traumatic spinal cord injury may be caused by an injury accident (e.g., a car accident), exercise (e.g., an accident during exercise, an accident during diving, etc.), or may be caused by bone fraction, hematoma, or the pressure on a disc material. The non-traumatic spinal cord injury includes arthritis, degenerative joint disease, vertebral subluxation, myelitis, syringomyelia, and spinal tuberculosis, but is not limited thereto. For example, the myelitis includes acute transverse myelitis, acute disseminated myelitis, myelopathy, non-Hodgkin's lymphoma, hydrocephalus, neurosyphilis, Minamata disease, Lou Gehrig's disease, and multiple sclerosis. Meanwhile, the spinal cord injury may also involve, for example, a ligament in the cervical vertebrae, pleural effusion, lumbosacral area, conus medullaris, larynx, or cauda equine.

According to one example of the present invention, when human ESC-derived neural precursor cells (NPCs) were administered into spinal cord injury test models (SCI models), a group transplanted with pluripotent stem cells (ESC)-derived neural precursor cells showed excellent behavior recovery in the BBB test (see FIG. 2). The induced pluripotent stem cells (iPSCs) are considered to be identical to the pluripotent stem cells (e.g., embryonic stem cells) since the iPSCs have stem cell gene and protein expression patterns, chromosomal methylation patterns, doubling time, embryoid body formation, teratoma formation, viable chimera formation, hybridizability, and differentiability.

The treatment method of the present invention encompasses the treatment for spinal cord injury through the administration (or transplantation) of a therapeutically effective amount of PSA-NCAM positive neural cells together with at least one therapy or treatment method. The additional therapy may be employed before, at the same time of, or after the administration of PSA-NCAM positive neural cells. For example, the treatment method of the present invention includes a surgical stabilization through the insertion of a stick or screw, in order to suitably arrange the spine or fusing adjacent vertebrae to strengthen the vertebrae, promote bone re-growth, and reduce the future possibility of additional SCI. Alternatively, the treatment of the present invention includes functional electric stimulation (FES) of particular nerves or muscles.

In cases where the composition of the present invention is prepared as a pharmaceutical composition, the pharmaceutical composition of the present invention contains a pharmaceutically acceptable carrier. The pharmaceutically acceptable carrier contained in the pharmaceutical composition of the present invention is one that is conventionally used in the formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, saline, phosphate buffered saline (PBS), and media.

The pharmaceutical composition of the present invention may further contain, in addition to the above ingredients, a lubricant, a wetting agent, a sweetening agent, a flavoring agent, an emulsifier, a suspending agent, a preservative, and the like. Suitable pharmaceutically acceptable carriers and preparations are described in detail in Remington's Pharmaceutical Sciences (19th ed., 1995).

The pharmaceutical composition of the present invention may be administered orally or parenterally. The parenteral administration is an intramuscular administration, an administration into a site of injury (damage), an intraspinal or intrathecal administration, or an intravascular administration.

A suitable dose of the pharmaceutical composition of the present invention may vary depending on various factors, such as the method for formulation, the method of administration, the age, body weight, gender, morbidity, and diet of the patient, time of administration, route of administration, excretion rate, and response sensitivity. The general dose of the pharmaceutical composition of the present invention is 10²-10¹⁰ cells per day on the basis of an adult.

The pharmaceutical composition of the present invention may be administered once or multiple times a day. For example, the pharmaceutical composition of the present invention may be administered to human or other animals as one dose or in multiple divided doses. The single-dose composition may fill a daily dose by containing a predetermined range of amount or a content corresponding to a portion thereof. That is, the composition for treatment according to the present invention may be administered to a patient in need of a treatment once or multiple times a day, or may be administered once or multiple times at intervals of a predetermined time (hour, day, week, etc.).

The pharmaceutical composition of the present invention may be formulated into a unit dosage form or may be prepared in a multi-dose container by using a pharmaceutically acceptable carrier and/or excipient according to the method easily conducted by a person having an ordinary skill in the art to which the present invention pertains. Here, the dosage form may be a solution in an oily or aqueous medium, a suspension, a syrup, or an emulsion, an extract, a pulvis, a powder, a granule, a tablet, or a capsule, and may further include a dispersant or a stabilizer.

### Advantageous Effects

Features and advantages of the present invention are summarized as follows:

(i) The present invention provides a method for treating spinal cord injury, the method comprising administering a composition, which contains poly-sialylated neural cell adhesion molecule (PSA-NCAM)-positive neural cells as an active ingredient, to a subject in need thereof.

(ii) The PSA-NCAM positive neural cells (e.g., PSA-NCAM positive neural precursor cells) of the present invention, when transplanted into spinal cord injury animal models, are differentiated into astrocytes, oligodendrocytes, or neurons (NeuN positive cells), which are three important cell types constituting neural tissues, exhibiting a behavior improvement effect due to the recovery of neural functions and the restoration of the injured spinal cord tissues, and thus the cells can be used for a composition for preventing, alleviating, or treating spinal cord injury.

### BRIEF DESCRIPTION OF THE DRAWINGS

FIG. 1 shows the construction of a spinal cord injury model and a schematic view of a cell transplantation procedure.

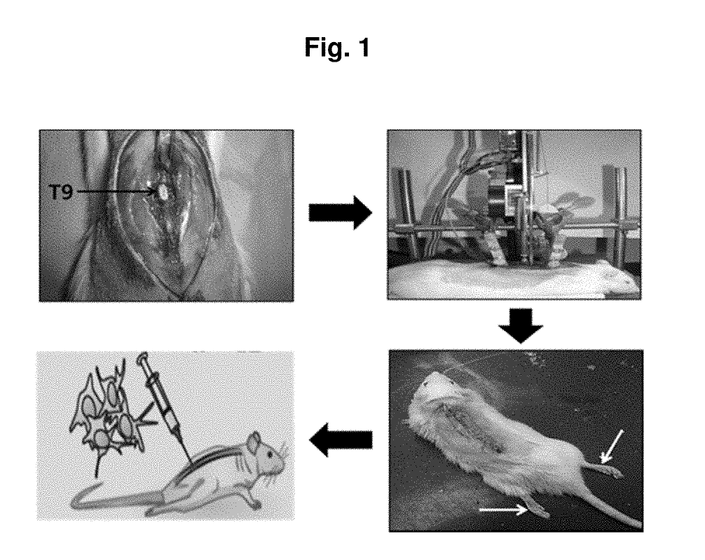

FIG. 2 shows that a cell transplantation group showed excellent behavior recovery compared with a PBS treatment control group when spinal cord injury models were subjected to the BBB test for 10 weeks (P<0.05).

FIG. 3 shows immunohistochemical results of the PBS treatment control group and the cell transplantation groups. Cell viability and differentiation were observed in tissues 10 weeks after transplanting PSA-NCAM positive cells to spinal cord injury animal models.

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### Examples

### Materials and Methods

Preparation of Human ESC-Derived or Human iPCS-Derived NPCs and PSA-NCAM Positive NPCs

The use of human cells was approved by the Institutional Review Board (IRB No. 4-2008-0643). For neural induction, embryoid bodies (EBs) derived from hESCs and iPSCs were cultured for 4 days in suspension with 5 μM dorsomorphin (DM) (Sigma, St. Louis, Mo.) and 5-10 μM SB431542 (Calbiochem, trogen) in hESC media deprived of bFGF (Invitrogen), and then attached on Matrigel-coated dishes (BD Biosciences, Bedford, Mass.) in 1×N2 (Invitrogen) media supplemented with 20 ng/ml bFGF for the additional 5 days (Kim, D. S., Lee, D. R., Kim, H. S., et al. (2012) Highly pure and expandable PSA-NCAM-positive neural precursors from human ESC and iPSC-derived neural rosettes. PLoSOne, 7, e39715). Neural rosettes that appeared in the center of attached EB colonies were carefully isolated using pulled glass pipettes from the surrounding flat cells. Small rosette clumps were seeded on Matrigel coated dishes, and cultured in DMEM/F12 supplemented with 1XN2, 1XB27 (Invitrogen) (Kim, D. S., Lee, J. S., Leem, J. W., et al. (2010) Robust enhancement of neural differentiation from human ES and iPS cells regardless of their innate difference in differentiation propensity, Stem Cell Reviews and Reports, 6, 270-281).

Neural precursor cells (NPCs) composed of neural rosettes expanded upon reaching approximately 80-90% confluence were obtained, and then were exposed to 10 μM Y27632 (Sigma) for 1 hour to prevent cell-death prior to being subjected to MACS procedure. After dissociation using Accutase (Invitrogen), the cells (˜1×10⁸ cells) were blocked in 1% BSA-containing PBS, and then incubated together with anti-PSA-NCAM antibody conjugated with micro-beads (Miltenyi Biotec) at 4° C. for 15 minutes. After extensive washing, the cell suspension was loaded in magnetic activated cell sorting (MASC), and positively-labeled cells that remained in the column were eluted to a new tube with culture media after removing the column from the magnetic stand. The isolated PSA-NCAM positive neural precursor cells (NPCs) were plated at a concentration of 4-5×10⁵ cells/cm² in N2B27 media or NBG media supplemented with 20 ng/ml bFGF (1XN2, 0.5XB27, and 0.5XG21 supplement) (GeminiBio-Products, WestSacramento, Calif.). The culture media were changed every day and cells were passaged every 2-3 days.

Construction of Spinal Cord Injury Model

A two-week-old male Sprague-Dawley rat (body weight of about 250-300 g) was anesthetized, and then subjected to thoracic vertebrae 9 (Th 9) laminectomy (a procedure that removes vertebral bones surrounding the spinal cord). The thoracic vertebrae 9 bone of the spinal cord was removed through this microsurgery. Thereafter, the physical impact (impactor producing bruise) was artificially applied to the exposed spinal cord tissue, thereby inducing the spinal cord injury similar to that shown in humans. Spinal cord injury (SCI) was induced by dropping 10 g of a rod from a height of 25 mm onto the exposed spinal cord (from the removal of vertebrae bones on the surface) using the NYU impactor (spinal cord injury-causing machine). Lastly, after being favorably disinfected with Betadine, the wound was sutured. After the surgery, the rat was artificially urinated every day until the urinating function returned to normal.

Cell Transplantation

The BBB test (behavior recovery test) was performed on day 1, 4, and 7 after the spinal cord injury. After the BBB test was finished on day 7 (1 week), it was investigated whether the spinal cord injury model was favorably constructed (whether apparent hindlimb paralysis appeared). Only when the spinal cord injury model was favorably constructed, the experimental animal was re-anesthetized, and then the injured spinal cord was re-exposed for cell transplantation.

PSA-NCAM⁺ neural precursor cells differentiated from embryonic stem cells (ES cells) or induced pluripotent stem cells (iPS cells) were transplanted into the center region of the exposed spinal cord using a thinly drawn tube. PBS (control) or PSA-NCAM⁺ neural precursor cells (500,000 cells or 100,000 cells) were transplanted by directly blowing into the tube or using a syringe.

An immune suppressing drug was administered every day from one day before the cell transplantation.

Behavior Test (BBB Score Test) of Cell-Transplanted Spinal Cord Injury Model

The BBB test was performed for 10 weeks at weekly intervals from the day when the spinal cord injury rat model was transplanted with the cells. After the BBB test was performed for 10 weeks, comparison with the control was conducted.
Early Stage (score of 0-7): Composed of isolated joint movements with little or no hindlimb movement
Intermediate Stage (score of 8-13): Intervals of uncoordinated stepping
Late Stage (score of 14-21): Forelimb and hindlimb coordination

Immunohistochemistry of Cell-Transplanted Spinal Cord Injury Model

Upon completion of the behavior recovery test (10 weeks after cell transplantation or 11 weeks after spinal cord injury), the spinal cord tissues were removed from the rats through perfusion fixation, and then were subjected to immunohistochemistry. The removed spinal cord tissues were fixed with 4% formaldehyde for 24 hours, and washed with PBS. For paraffin section preparation, the tissues were dehydrated in cumulative ethanol, and paraffin-embedded. The paraffin-embedded tissues were cut into 10 um-thick layers on a microtome, deparaffinized in xylene for 10 minutes, and then rehydrated in cumulative alcohol. The segments were treated with 10 mM citric acid for 1 hour, and then 5% BSA solution containing PBS and 0.5% Triton X-100 was added. Thereafter, the spinal cord tissue segments were cultured at 4° C. for 15-17 hours together with primary antibodies against, as a neuron marker, HNA (human nuclear marker for grafted human cells Millipore, MAB1281, antibody concentration 1:100), Tuj1 (Covance, MMS-435P, antibody concentration 1:1000), MAP2 (Millipore, MAB3418, antibody concentration 1:200), or NeuN (Cell Signaling, 12943, antibody concentration 1:500); as an astrocyte marker, GFAP (Millipore, AB5804 antibody concentration 1:1000); as an oligodendroglia marker, NG2 (Millipore, AB5320, antibody concentration 1:200); as a neural precursor cell marker, Nestin (Millipore, ABD69, antibody concentration 1:2000); and as a proliferative cell marker, Ki67 (Leica Biosystems, NCL-KI67P, antibody concentration 1:150). The segments were cultured together with the primary antibody overnight, and then washed with PBS, and then the segments were cultured together with fluorescence-labeled secondary antibodies (Alexa-Fluor®488 or 594, 1:500, Molecular Probes, Eugene, Oreg., USA) for 1 hour. Fluorescence images of the segments were obtained using a fluorescence microscope (Olympus IX71).

Statistical Analysis

The statistical significance among groups was obtained using one-way analysis of variance (ANOVA) with Tukey's correction, and a p value<0.05 was determined to be statistically significant.

### Results

Behavior Recovery Test

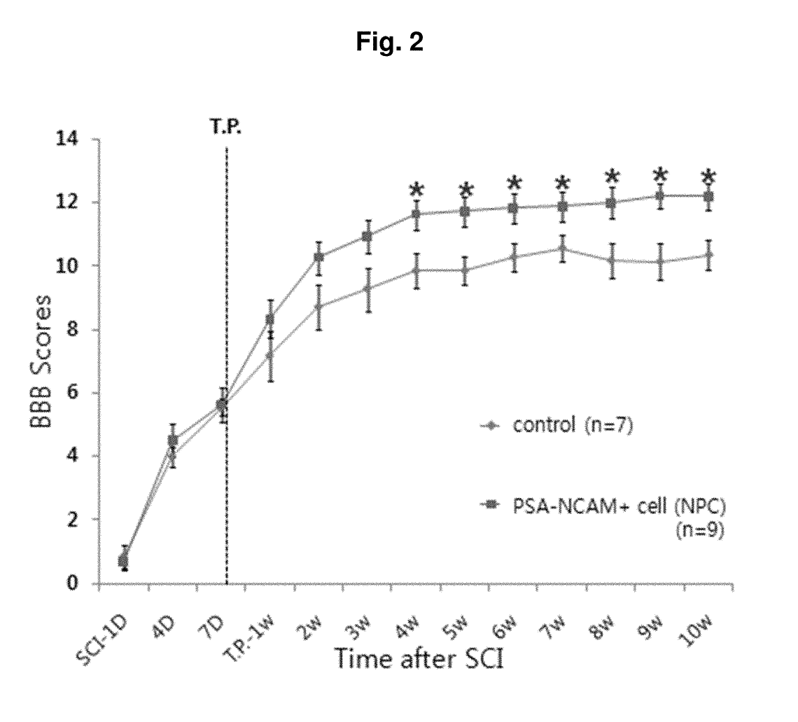

In order to understand the development of changes in the locomotor function through cell transplantation in the spinal cord injury (SCI) animal models, the Basso, Beattie, and Bresnahan (BBB) test was performed and scored every week by up to 10 weeks upon PSA-NCAM⁺ cell transplantation to SCI model animal (The cells were transplanted at 1 week after SCI model construction). As a result of checking the difference between groups, the cell transplantation group showed the improved locomotor function compared with a control group (PBS treatment group) (BBB score increase), and showed a statistically significant increase compared with the control group, from 4 weeks after transplantation (see FIG. 2, 95% significant level). T.P., Transplantation of cell.

Immunohistochemistry

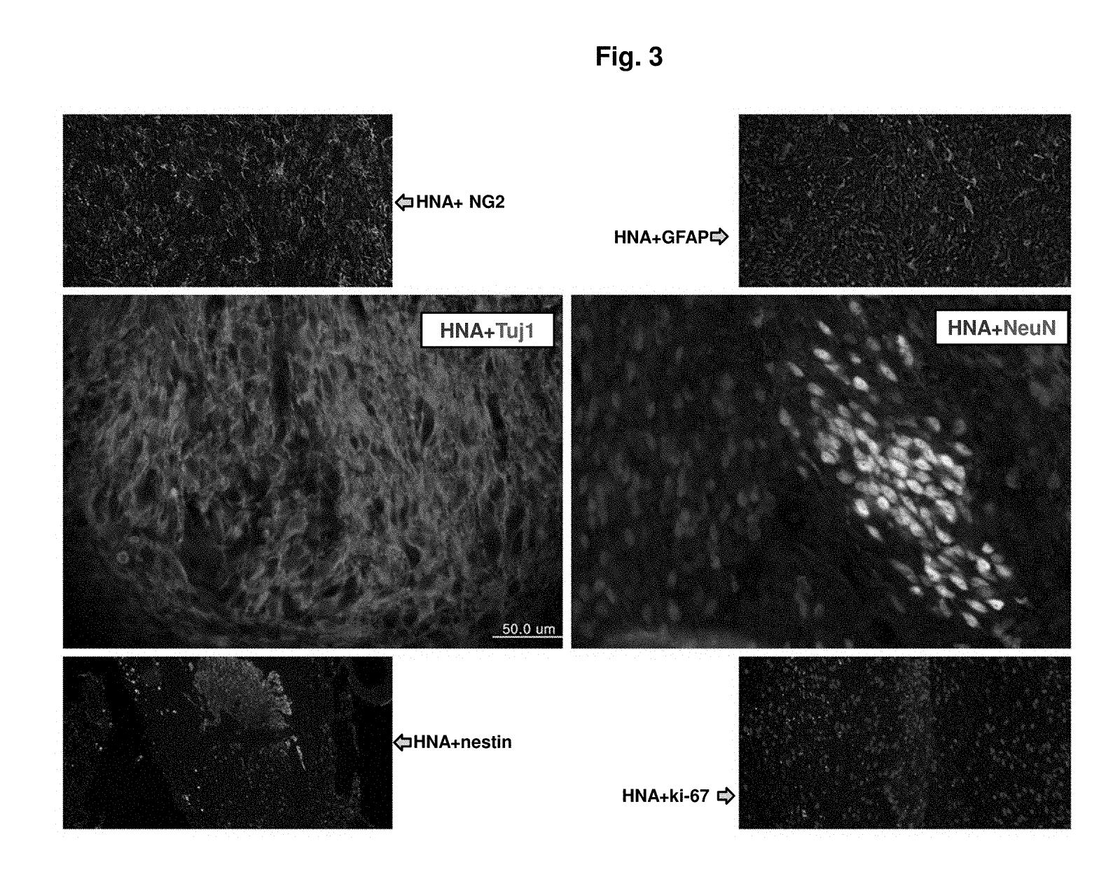

Cells were transplanted into the injured spinal cord tissues, and the spinal cord tissues were isolated at week 10, followed by immunohistochemical analysis. The transplanted PSA-NCAM⁺ cells appropriately survived, and then differentiated and distributed inside the spinal cord tissues for 10 weeks. Some of the undifferentiated neural precursor cells (nestin positivity) remained and are maintained. Hereinafter, the results were described with FIG. 3.

1. The rats were subjected to perfusion 10 weeks after cell transplantation and the BBS test.

2. Lots of GFAP positive astrocytes, NG2 positive oligodendrocytes, or Tuj1 positive neurons were observed in regions where many transplanted cells gathered (HNA, transplanted human cell marker). These results indicate that the transplanted PSA-NCAM+ cells (neural precursor cells) were favorably differentiated into astrocytes, oligodendrocytes, and neurons, which are three important cell types constituting neural tissues. It is determined that these differentiated neural cells were directly or indirectly involved in the restoration of the injured spinal cord tissues and thus the BBB scores are higher (improvement of the locomotor function) in FIG. 2. That is, the differentiated neural cells contributed to behavior recovery.

3. Cell nuclei were stained for HNA (red color), and cell nuclei were stained for NeuN (green color) as a mature neuron marker. In cases where the same site was stained for both markers, the composite of the two colors (i.e., yellow) was observed. The fact that the two markers were merged into one to display yellow accurately indicates that human-derived neural precursor cells (HNA positive cells) transplanted into the spinal cord injury animal models differentiated into neurons (NeuN positive cells).

4. It was verified that Nestin positive neural precursor cells were not yet differentiated and exist as cellular aggregates at predetermined areas, and this fact indicates that some of PSA-NCAM⁺ neural precursor cells were not differentiated into neurons, but still remain as neural precursor cells.

5. The proportion of Ki-67 positive cells (proliferating cells) was very low. This fact indicates that there are very few or no cells proliferating in the transplanted tissues.

Characteristic of Used Antibodies:

1. HNA (human nuclear marker)—indicating that the cells transplanted into the rat are human-derived cells

2. Tuj1—indicating that the neural precursor cells are differentiated into neurons (neuron marker).

3. NeuN (neuron markers)—indicating that neural precursor cells are differentiated into neural cells.

4. GFAP (astrocyte marker)—indicating that neural precursor cells are differentiated into astrocytes

5. NG2 (oligodendrocyte marker)—indicating that neural precursor cells are differentiated into oligodendrocytes

6. Nestin (neural precursor cell marker)—indicating that some of neural precursor cells may still remain without differentiation.

7. Ki67 (proliferating cell marker) indicating proliferating cells among the transplanted cells

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### REFERENCES

1. Caplan, A. I., & Dennis, J. E. (2006). Mesenchymal stem cells as trophic mediators. Journal of Cellular Biochemistry, 98, 1076-1084.
2. Chen, J., Li, Y., Katakowski, M., et al. (2003). Intravenous bone marrow stromal cell therapy reduces apoptosis and promotes endogenous cell proliferation after stroke in female rat. Journal of Neuroscience Research, 73, 778-786.
3. Kopen, G. C., Prockop, D. J., & Phinney, D. G. (1999). Marrow stromal cells migrate throughout forebrain and cerebellum, and they differentiate into astrocytes after injection into neonatal mouse brains. Proceedings of the National Academy of Sciences, 96, 10711-10716.
4. Liu, J., Han, D., Wang, Z., et al. (2013). Clinical analysis of the treatment of spinal cord injury with umbilical cord mesenchymal stem cells. Cytotherapy, 15, 185-191.
5. Bhasin, A., Srivastava, M. V., Mohanty, S., Bhatia, R., Kumaran, S. S., & Bose, S. (2013). Stem cell therapy: a clinical trial of stroke. Clinical Neurology and Neurosurgery, 115, 1003-1008.
6. Bae, K. S., Park, J. B., Kim, H. S., Kim, D. S., Park, D. J., & Kang, S. J. (2011). Neuron-like differentiation of bone marrow-derived mesenchymal stem cells. Yonsei Medical Journal, 52, 401-412.
7. Cho, S. R., Kim, Y. R., Kang, H. S., et al. (2009). Functional recovery after the transplantation of neurally differentiated mesenchymal stem cells derived from bone barrow in a rat model Functional recovery after the transplantation of spinal cord injury. Cell Transplantion, 18, 1359-1368.
8. Capone, C., Frigerio, S., Fumagalli, S., et al. (2007). Neurosphere-derived cells exert a neuroprotective action by changing the ischemic microenvironment. PLoSOne, 7, e373.
9. Kim, D. S., Lee, D. R., Kim, H. S., et al. (2012). Highly pure and expandable PSA-NCAM-positive neural precursors from human ESC and iPSC-derived neural rosettes. PLoSOne, 7, e39715.
10. Honmou, O., Onodera, R., Sasaki, M., Waxman, S. G., Kocsis, J. D. (2012). Mesenchymal stem cells: therapeutic outlook for stroke. Trends in Molecular Medicine. 18, 292-297.
11. Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131: 861-872.

1. A method for treating spinal cord injury, comprising administering a composition, which comprises poly-sialylated neural cell adhesion molecule (PSA-NCAM)-positive neural cells as an active ingredient, to a subject in need thereof.

2. The method of claim 1, wherein the neural cells are PSA-NCAM positive neural precursor cells, a PSA-NCAM positive neuronal precursor cells, or differentiated PSA-NCAM positive neural cells.

3. The method of claim 2, wherein the neural cells are PSA-NCAM positive neural precursor cells.

4. The method of claim 3, wherein the neural precursor cells are neural precursor cells before or after the neural rosette stage, differentiated from pluripotent stem cells.

5. The method of claim 1, wherein the neural cells are differentiated from pluripotent stem cells.

6. The method of claim 5, wherein the pluripotent stem cells are embryonic stem cells, induced pluripotent stem cells (iPSCs), embryonic germ cells, or embryonic carcinoma cells.

7. The method of claim 1, wherein the spinal cord injury is a traumatic spinal cord injury or non-traumatic spinal cord injury.

8. The method of claim 7, wherein the traumatic spinal cord injury is caused by an injury accident including a car accident, an accident during exercise and an accident during diving; bone fraction; hematoma; or the pressure on a disc material.

9. The method of claim 7, wherein the traumatic spinal cord injury is a flexion injury, vertical compression injury, hyperextension injury, or flexion rotation injury.

10. The method of claim 7, wherein the non-traumatic spinal cord injury is arthritis, degenerative joint disease, vertebral subluxation, myelitis, syringomyelia, spinal cord inflammation, inflammatory spinal cord injury or spinal tuberculosis.
